(19) United States
(12) Patent Application Publication
(10) Pub. No.: US 2005/0089512 A1
(43) Pub. Date: Apr. 28, 2005

(54) Skin/hair equivalent with reconstructed papillae

(76) Inventors: Kordula Schlotmann, Dusseldorf (DE); Thomas Gassenmeier, Dusseldorf (DE); Ralf L. Paus, Hamburg (DE); Melanie Giesen, Geldern (DE); Dirk Petersohn, Koln (DE)

Correspondence Address: WOODCOCK WASHBURN LLP, ONE LIBERTY PLACE, 46TH FLOOR, PHILADELPHIA, PA, 19103, US

(21) Appl. No.: 10/871,119

(22) Filed: Jun. 18, 2004

Related U.S. Application Data
(63) Continuation of application No. PCT/EP02/14212, filed on Dec. 13, 2002.

(30) Foreign Application Priority Data
Dec. 19, 2001 (DE) DE 101 62 814.5

(51) Int. Cl. C12N 5/08; A61K 45/00

(52) U.S. Cl. 424/93.7; 435/371

(57) ABSTRACT

The invention relates to a skin/hair equivalent, more particularly a hair model with reconstructed papillae (pseudopapillae; PP) in a reconstructed dermis (pseudodermis; PD), to its production and to its use, more particularly for medical/pharmaceutical purposes and for application in the cosmetics industry.

### CROSS REFERENCE TO RELATED APPLICATIONS

This application is a continuation of PCT/EP02/14212 filed Dec. 13, 2002, which claims the benefit of DE 101 62 814.5, filed Dec. 19, 2001, the complete disclosures of which are hereby incorporated by reference in their entirety.

### FIELD OF THE INVENTION

This invention relates to a skin/hair equivalent, more particularly a skin/hair model with reconstructed papillae (pseudopapillae) in a reconstructed dermis (pseudodermis), to its production and to its use, more particularly in the medical, pharmaceutical and cosmetics fields.

Finding active substances with, for example, a biological effect on the hair follicle, so that they are capable of influencing hair pigmentation, hair growth and hair structure, requires suitable in vitro test systems on which any such effect can be evaluated. These test systems should ideally allow the screening of a relatively large number of substances, should be standardizable and inexpensive and—in the case of in vitro systems—should simulate the in vivo situation.

In hair research, there are at present no suitable in vitro models, for example for studying hair growth, hair pigmentation and hair structure. A summary of existing methods and an illustration of the disadvantages of these systems can be found, for example, in K. S. Stenn “Laboratory Assessment of Hair Follicle Growth” in Skin Pharmacol. Appl. Skin Physiol. 1999; 12: 154-157. These systems range from monolayer cell cultures via animal models and ex-vivo systems to in vitro systems.

Monolayer cultures of hair follicle cells have the disadvantage that, when removed from their complex three-dimensional structures, the cells behave differently than they would in the organ as a whole. Because of this, information on the effect of substances on hair follicle cells cultivated as a monolayer is of little relevance to the in vivo situation. Under the guidelines on cosmetics, animal models may not be used for the development of cosmetic products. Accordingly, ex vivo models which combine in vitro methods with in vivo methods on the animal are also out of the question. Similar problems as to the availability of material and standardizability are involved in the use of skin explantates with hairs in culture. Although in vivo studies on human beings are carried out to screen the effect, they are not advisable until a potent active substance has been discovered and incorporated in a formulation because such studies are correspondingly expensive and complex. There are also no suitable in vitro test systems for screening substances which influence hair color.

In vitro tests for modifying hair pigmentation by influencing the melanin production of the melanocytes are also mainly carried out on single cell cultures. Epidermal melanocytes or B16 melanoma cells are often used for this purpose and the results obtained with this cell type are extrapolated to the hair melanocytes because hair melanocytes are difficult to isolate and cultivate. In addition, the complex interaction of the melanocytes with the hair follicle is missing in these systems so that their relevance to the situation in vivo on the hair follicle has to be called into question. The same applies to reconstructed hair models which contain (epidermal) melanocytes. Animal models which are also a popular test model for substances with an effect on hair pigmentation are prohibited under the guidelines on cosmetics where the substances are to be used for cosmetic products. In vivo studies on human beings are laborious and expensive and, accordingly, are only advisable after a potent active substance has been found.

In the technique of tissue engineering, various cell types are isolated from tissue, for example skin tissue, and multiplied in cell culture as a so-called monolayer. The tissue is then reconstructed from the single cells. In the case of skin, fibroblasts, for example, can be “sown” into a collagen gel or other matrix so that they proliferate and form a pseudodermis. Epidermal keratinocytes can be applied to the pseudodermis thus formed where they also proliferate and form a pseudoepidermis. By raising the culture into the air (air/liquid interface), the cells begin to differentiate and to form a stratum corneum.

Hitherto, cell cultures, for example keratinocytes of the outer root sheath (ORS keratinocytes, ORS=outer root sheath), dermal papilla cells, etc., have mainly been used in hair research. However, repeated attempts have also been made to cultivate hair follicles or parts of hair follicles as a three-dimensional model, for example in a collagen gel, or to reconstruct entire hair follicles by combining different hair follicle cells.

In “Characterization of a new tissue-engineered human skin equivalent with hair” published in In vitro Cell. Dev. Biol. Animal 35:318-326, June 1999, M. Michel et al. report for the first time on the insertion of a hair follicle into a reconstructed hair model for use in penetration studies. Here, the authors used whole hair follicles which had to be prepared beforehand from hair-covered skin. Apart from the limited availability of the material, standardizability is poor where prepared hairs are used because the biological variations are considerable.

EP 0 285 471 A1 and EP 0 285 474 A1 also describe the production of an artificial skin which consists of a dermal layer of contractile cells (fibroblasts) and extracellular matrix components into which whole hair follicles or follicle segments are inserted. The dermal layer is then additionally coated with keratinocytes which form an epidermal layer. The disadvantage here is that the papillae are not reconstructed, instead only part of the hair follicle with no papilla is used.

The model used by a group of Japanese researchers (M. Inamatsu et al. “Hair Follicle Development in Organotypic Culture”, Third Intercontinental Meeting of Hair Research Societies (Abstract), Tokyo, 2001) for the early phase of hair development consists of freshly isolated dermal papillae from rat whiskers which are inserted between a collagen gel containing fibroblasts and an epidermal layer of rat keratinocytes. This organotypic culture is cultivated at the air/liquid interface. After 7 days, the epidermis is said to thicken in the vicinity of the dermal papillae. Firstly, no human cells or papillae are used here, secondly the papillae are not reconstructed papillae but whole papillae isolated from hair follicles and thirdly the papillae are not inserted (for example injected or grafted) into the epidermis, but are placed between the dermal and the epidermal layers. The use of isolated papillae involves the same problems of availability and standardizability as the use of isolated hair follicles.

The same applies to the works of S. A. J. Watson et al. “Sheep vibrissa dermal papillae induce hair follicle formation in heterotypic skin equivalents” in British Journal of Dermatology (1994) 131: 827-835. Here, dermal papillae or papilla cells cultivated in a collagen gel are placed between the dermis and epidermis of a skin equivalent with a view to obtaining a model for the development of the hair follicle. Under these conditions, however, the dermal papilla cells migrate into the dermal matrix and do not form themselves into a papilla so that the model has to be modified by applying the papillae or the papilla cells to a dermal substrate and which is then covered by a fetal mouse epidermis and transplanted onto hairless mice.

In 1993, A. B. Jahoda et al. “Dermal-Epidermal Interactions—Follicle-Derived Cell Populations in the Study of Hair-Growth Mechanisms” in J. Invest. Dermatol. 101: 33S-38S, 1993 succeeded in producing a follicle-like structure from a combination of hair cells by first cultivating outer root sheath cells (ORS cells) in the collagen capsule of the follicle of a rat whisker and then adding a mixture of different hair follicle cells—dermal papilla cells, dermal sheath cells and matrix cells. However, they needed the stable collagen capsule of the rat whisker to stabilize the structure.

A. Limat et al. “Outer Root Sheath (ORS) cells organize into epidermoid cyst-like spheroids when cultured inside Matrigel®: a light-microscopic and immunohistological comparison between human ORS cells and interfollicular kera-tinocytes” in Cell Tissue Res. (1994) 275: 169-176 also fit various hair follicle cells and skin cells together in a three-dimensional structure in order to study their interaction. They use a collagen (I) gel as base and, on it, bed a layer of Matrigel® or a mixture of basal membrane components containing various cells or cell mixtures. In the Matrigel®, the epidermal or ORS keratinocytes (=Outer root Isheath keratinocytes) form spheroidal, but non-follicular structures. Here, layers containing various cells are placed one above the other. However, no new structure is built up in these layers like the dermal papilla. Although Limat et al. report that ORS keratinocytes are capable of forming cystoidal structures which turn horny internally, it is not a question here of the physiological nature of hair shaft formation, but rather of the differentiation and stratum corneum formation by cystoidally arranged keratinocytes. However, hair shafts are normally formed by matrix cells which lie above the dermal papilla.

Published Japanese patent application 10-136977 (Toyobo Co., Ltd., Japan) describes an artificial tissue and its reconstruction which comprises hair-shaft-like structures at the boundary between a layer of fibroblasts in a collagen layer and a collagen layer containing hair papilla cells. This model is said to serve as a test system for determining the compatibility and effectiveness of active substances and cosmetics. The same applies here as to the article by A. Limat et al.: in this model, dermal papillae which share the three-dimensional structure of the hair follicle are not reconstructed, instead the cells are arranged one above the other in layers and no new structure is formed. Melanocytes are apparently not used in the described model either. Effectiveness tests are mentioned as a potential application for the model. However, there is no indication of which end points are to be evaluated on the model, i.e. how the application of active substances affects the structure of the reconstructed model and what can be read into this so far as the effect of this substance on hair growth and hair structure are concerned.

The “Philpott Model” (M. P. Philpott “Human hair growth in vitro”, J. Cell. Sci. 97, 463-471,1990), where isolated hair follicles are kept in culture for 9 days, has the disadvantage on the one hand of significant variability between the individual follicles and hence poor standardizability and, on the other hand, poor availability of the hair follicles. Because of this, only a very limited amount of active substances can be evaluated within a fixed period. In addition, only substances and formulations which are soluble in the medium can be applied, but not water-insoluble substances or formulations, such as creams for example.

In what was virtually a further development of the Philpott Model, an attempt was also made to insert isolated hair follicle segments into reconstructed skin models (see M. Inamatsu et al. and M. Michel et al.). Although follicles, follicle segments or dermal papillae are thus in contact with the dermis, which comes closer to the in vivo situation than other known models do, the disadvantages of these systems where they are to be used for studying active substances are similar to those attending the Philpott Model (poor availability of the hair follicles, no standardizability, high cost, etc.).

### SUMMARY OF THE INVENTION

Accordingly, the problem addressed by the present invention was to provide a skin/hair equivalent (“skin model”), more particularly a hair/skin model with reconstructed papillae in a reconstructed dermis, which would at least partly avoid the above-mentioned disadvantages of the prior art.

Another problem addressed by the present invention was to find or provide a skin/hair equivalent or model which would be suitable as an in vitro model system, more particularly for testing and/or evaluating active substances, more particularly on the hair follicle. Such a model would be particularly suitable for discovering and testing pharmaceutical/medical and cosmetic active principles. It would also allow in vitro evaluation of the effect of such active principles on the hair follicle, hair growth, hair pigmentation, hair structure and the like.

Accordingly, the present invention relates to a process for the production of a skin/hair equivalent, more particularly a skin/hair model with reconstructed papillae (pseudopapillae; PP) in a reconstructed dermis (pseudodermis; PD), the process comprising the following steps:
(a) providing a suitable reconstructed dermis (pseudodermis; PD) or a pseudodermis preparation;
(b) providing reconstructed papillae (pseudopapillae; PP) comprising cultivated papilla cells, preferably dermal papilla cells (hair papilla cells), in a suitable matrix, more particularly gel matrix, or providing corresponding precursors of such reconstructed papillae (pseudopapillae; PP) comprising cultivated papilla cells, more particularly dermal papilla cells (hair papilla cells), in a suitable matrix-forming, more particularly gel-forming,
medium MFMₚₚ which is capable of forming a matrix, more particularly a gel matrix, in situ, more particularly in the reconstructed dermis (pseudodermis; PD);
(c) introducing or inserting the reconstructed papillae (PP) or their precursors provided in step (b) into the pseudodermis (PD) or the pseudodermis preparation provided in step (a); and (d) optionally applying a reconstructed epidermis (pseudoepidermis; PE) or a reconstructed periderm (pseudoperiderm; PI) to the pseudodermis (PD).

### BRIEF DESCRIPTION OF THE DRAWINGS

FIG. 1 is a schematic illustration of one particular embodiment of the process according to the invention. A pseudodermis (PD) based on fibroblasts in a matrix of type I collagen is shown at (1). Shown at (2) is a precursor of a pseudopapilla consisting of a mixture of the cultivated dermal papilla cells and at least one matrix former MFₚₚ, from which the actual pseudopapillae (PP) can then be formed, for example by direct injection, so that this mixture then forms the matrix and hence the pseudopapillae (PP) in situ in the pseudodermis (PD), more particularly by gelling. The reconstructed papilla model shown at (3) is formed.

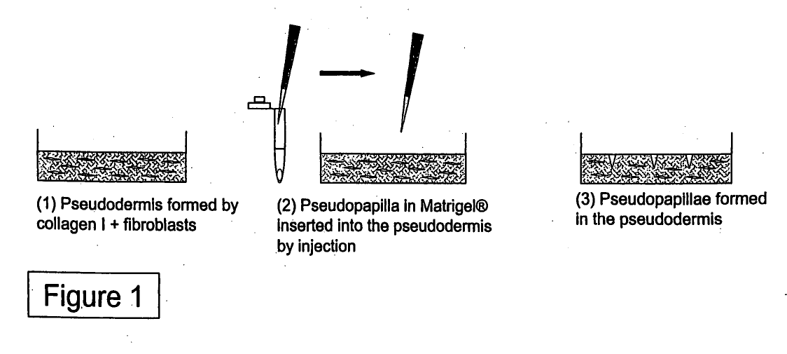

FIG. 2 is a schematic illustration of another particular embodiment of the process according to the invention. Shown at (1) is a pseudodermis (PD) based on fibroblasts in a matrix of type I collagen, into which cavities or openings for accommodating the pseudopapillae (PP) have been punched, the cavities optionally being lined by spraying with type IV collagen or Matrigel®. A pseudopapilla consisting of a gelatin-based microcarrier and dermal papilla cells grown thereon is shown at (2). At (3), the pseudopapilla (PP) formed from microcarriers are introduced or inserted (for example by injection) into the pseudodermis (PD). The reconstructed papilla model shown at (4) is formed.

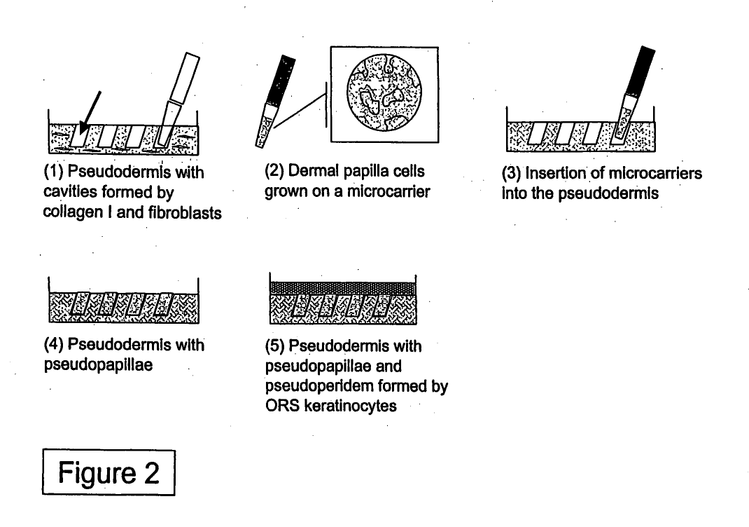

Follicle-like or follicular structures, including those reminiscent of the earliest stages of hair morphogenesis (morphogenesis stages I to III), such as the periderm for example, are then formed in this three-dimensional hair/skin model at (5).

### DETAILED DESCRIPTION OF ILLUSTRATIVE EMBODIMENTS

Accordingly, step (a) of the process comprises providing a reconstructed dermis or pseudodermis or a pseudodermis preparation. According to the invention, any pseudodermis known from the prior art may be used providing it is suitable for the process according to the invention which means in particular that it is compatible with the other constituents of the skin/hair equivalent according to the invention. In particular, the pseudodermis (PD) used in accordance with the invention comprises cultivated contractile cells, more particularly fibroblasts, preferably dermal fibroblasts, in a suitable matrix. The matrix may be, in particular, a matrix based on collagen, preferably type I and/or type III collagen, and optionally other components. The cultivated contractile cells for the pseudodermis (PD) or the pseudodermis preparation may be obtained in known manner, for example by isolating dermal fibroblasts from human or animal skin and, after cultivation, recovering the contractile cells from the resulting monolayer cultures (for example by moderate trypsinization). A suitable nutrient medium which should be compatible with the contractile cells in particular is used for cultivating those cells. According to the invention, one example of a suitable nutrient medium is essential minimal medium MEM (Modified Eagle Medium).

The pseudodermis (PD) may then be obtained by mixing the contractile cells recovered from monolayer cultures and optionally present in a suitable nutrient medium with a matrix-forming, more particularly gel-forming, medium MFMPD which contains at least one matrix former MFPD, more particularly gel former, and optionally other constituents.

In the context of the invention, the resulting mixture is referred to as the pseudodermis preparation. Depending on the concentration of the matrix-forming medium, the pseudodermis preparation forms a matrix, preferably a gel matrix, relatively quickly (higher concentration of the matrix-forming medium) or relatively slowly (lower concentration) and finally contracts, optionally with ejection of any nutrient medium present, to a pseudodermis (PD). All phases or states of the matrix forming process and the contraction process are encompassed by the term “pseudodermis preparation”. Accordingly, the pseudodermis is obtained on completion of the matrix formation and contraction of the pseudodermis preparation.

The matrix former MF_{PD}, more particularly gel former, of the matrix-forming, more particularly gel-forming, medium MFM_{PD} may be in particular a matrix former based on collagen, preferably type I and/or type III collagen, and optionally other components (for example constituents of the extracellular matrix of the dermis, preferably matrix and/or scleroproteins, such as laminin).

A suitable nutrient medium, more particularly essential minimal medium MEM, and optionally other components may optionally be applied to the pseudodermis (PD) thus provided or generated.

The pseudopapillae (PP) provided in step (b) comprise cultivated papilla cells, more particularly dermal papilla cells (hair papilla cells), on a suitable carrier and/or in a suitable matrix. The matrix may be in particular a matrix based on collagen, more particularly type IV collagen, and optionally other components. The cultivated dermal papilla cells (hair papilla cells) may be produced in known manner, for example by isolating dermal papilla cells (hair papilla cells) from the hair follicles of human or animal skin and, after cultivation, recovering the dermal papilla cells from the resulting monolayer cultures, more particularly by moderate trypsinization. The number of passages should be small, the number of passages for the dermal papillae generally being between 1 and 10 and preferably between 1 and 3. The dermal papilla cells are cultivated in a suitable nutrient medium, the nutrient medium used being in particular essential minimal medium MEM, for example DMEM (Dulbecco's Modified Eagle Medium) and/or RPMI medium (medium developed by the Roswell Park Memorial Institute) and/or Chang medium, optionally together with other components, for example fetal calf serum (FCS), collagen, more particularly type I collagen, and the like. Examples of nutrient media suitable in accordance with the invention for cultivating the dermal papilla cells are any RPMI— or DMEM-based cell culture media such as, for example, RPMI 1640 (Sigma) containing 20% FCS, Chang medium (Irvine Scientific) containing 10% FCS and the like.

The pseudopapillae (PP) are then obtained by mixing the papilla cells recovered from monolayer cultures and optionally present in a suitable nutrient medium with a matrix-forming, more particularly gel-forming, medium MFMₚₚ which contains at least one matrix former MFₚₚ, more particularly gel former, and optionally other constituents; the resulting mixture forms a matrix, preferably a gel, and then contracts with ejection of the nutrient medium present, if any. The pseudopapillae (PP) can then be formed (for example by punching or cutting out) from the contracted matrix or the contracted gel.

In a particularly preferred embodiment, the pseudopapillae (PP) are produced by growing papilla cells, more particularly dermal papilla cells, on a suitable carrier. Cell groups already interacting can thus advantageously be produced in a manner similar to the papilla.

The carrier may be both porous and non-porous and may have no pores or small or large pores. A porous surface is preferred so that the papilla cells are better able to imitate the natural three-dimensional aggregates. A swelling carrier material (for example polysaccharide- or polypeptide-based) or a non-swelling carrier material (for example polymers) may also be used.

The carrier size (or particle size, i.e. the diameter or the greatest extent in one dimension) is preferably in the range from about 50 to 2,000 μm and more preferably in the range from 100 to 1,000 μm. Particle sizes of about 100 to 500 μm are particularly preferred because, on the one hand, handling ability in the production of the pseudopapilla and, on the other hand, a sufficiently high similarity to an actual hair papilla are guaranteed. Suitable carriers are in particular the so-called microcarriers, small globular particles on which cells grow in three-dimensional geometry.

Carriers with any three-dimensional form may be used. Spherical, conical or cylindrical and polyhedral, round or ellipsoidally flattened or polygonal (for example hexagonal) carriers are suitable, spherical carriers being particularly preferred because they are able to imitate the shape of the papilla particularly well.

The carrier may consist of inorganic and/or organic material which may be both modified and unmodified at its surface.

Surface modifications may be physical in nature, for example the coating of the carrier to prevent the adhesion of particles, for example steel particles or polymers (more particularly biopolymers, preferably polypeptides and/or polysaccharides). Surface modification with matrix-forming proteins, more particularly collagen, preferably type I, III and IV collagen, and matrix and/or scleroproteins (preferably laminin, gelatine, chitosan, glucosamines, glycosaminoglycans (GAG), heparan sulfate proteoglycans, sulfated glycoproteins, such as nidogen (more particularly entactin), tissue plasmimogen activator, Matrigel® and mixtures of the above-mentioned constituents.

However, the surface can also be modified by chemical modification. For example, various surface charges can be obtained if the surface is not already charged from the carrier material itself. Carriers positively charged by surface modification of a crosslinked dextran matrix with N,N-diethylaminoethyl groups are mentioned by way of example.

Glasses, silicones or polymer matrices, for example polystyrenes, etc., are suitable. Matrices of polysaccharides, such as dextran, or polypeptides, such as gelatine, are particularly suitable. Crosslinked polymers, such as crosslinked gelatin (especially highly crosslinked gelatin, for example Cultisphere®, Percell) or crosslinked dextran, are also particularly suitable.

The pseudopapilla may be produced simply by adding the carrier to the papilla cell culture. Incubation times of one to several days or even several weeks can be suitable, depending on the cell growth density. Thorough but not overly intensive mixing of the culture medium is preferred in order to ensure optimal cell growth on the carrier surface. For example, gentle stirring or shaking or even cultivation in fluidized bed or stirred reactors or spinner bottles are suitable.

The pseudopapilla formed from the carrier covered with papilla cells may then be embedded in the prepared pseudodermis or the pseudodermis preparation.

As described in the following, however, the pseudopapillae (PP) may also be formed in situ, more particularly in the pseudodermis (PD). The matrix-forming, more particularly gel-forming, medium MFMₚₚ contains as matrix former MFMₚₚ more particularly gel former, at least one collagen, more particularly type IV collagen, and optionally other constituents selected in particular from the group of matrix and/or scleroproteins, more particularly laminin, gelatin, chitosan, glucosamines, glycosaminoglycans (GAG), heparan sulfate proteoglycans, sulfated glycoproteins, such as nidogen (more particularly entactin), tissue plasmimogen activator and growth factors, such as tissue growth factor-beta (TGF-β), fibroblast growth factor, and other growth factors from the Engelbreth-Holm-Swarm Tumor (EHS Tumor) and/or human placenta and mixtures of the above-mentioned constituents. According to the invention, the following substances, for example, are suitable matrix materials for forming the pseudopapillae (PP): Matrigel® Basement Membrane Matrix (Matrigel®), collagen, gelatin, collagen/chitosan/GAG (GAG=glycosaminoglycan), glucosamine or any other type of matrix and mixtures of these substances. Collagen, Matrigel® and mixtures thereof are particularly preferred. Matrigel® and mixtures in a ratio of 0.1:1 to 10:1 (collagen:Matrigel®) are most particularly preferred.

As previously mentioned, the pseudopapillae (PP) may be formed from the matrix, more particularly the gel, the forming/shaping of the pseudopapillae (PP) taking place either before or after the introduction or insertion of the pseudopapillae (PP) or their precursors in step (c). The pseudopapillae formed by growing papilla cells on suitable carriers, whereby each carrier particle can be regarded as a pseudopapillae, may also be used in such a matrix in this way to produce a macroscopic pseudopapillae.

Both the matrix former MFPD for forming the pseudodermis (PD) and the matrix former MFₚₚ for forming the pseudopapillae (PP) should be capable of gelling on heating, more particularly at temperatures of 20° C. to 40° C., for example polymerizing in the process, and of promoting the growth and differentiation of cells. The matrix of the pseudodermis (PD) and the matrix of the pseudopapillae (PP) are generally formed in three-dimensional structures. Formation of the matrix, more particularly the gel, can be reversible or irreversible although it is preferably irreversible.

The introduction or insertion of the pseudopapillae (PP) in step (c) can be carried out in various ways. In one embodiment, suitable cavities for accommodating the pseudopapillae (PP) are first formed, preferably in the already contracted pseudodermis (PD), more particularly by punching or pricking, and the pseudopapillae (PP), which contain cultivated papilla cells and which are so shaped that their dimensions correspond to the cavities formed in the pseudodermis (PD), are then introduced or inserted (for example by grafting) into those cavities. Punching of the pseudodermis (PD) or pricking of the pseudodermis (PD) may be carried out, for example, with a punch (for example with a diameter of 0.5 to 4 mm, preferably about 2 mm) or with a button cannula or with a conventional cannula.

Before introduction of the pseudopapillae (PP) or their precursors, the cavities formed by punching or pricking of the pseudodermis (PD) may be lined (for example by spraying), more particularly with at least one collagen, preferably type IV collagen, and/or other matrix proteins, more particularly basal membrane proteins, such as laminin. The cavities are preferably lined with mixtures of two or more of the constituents mentioned, more particularly Matrigel®. Where this procedure is adopted, the pseudopapillae (PP) are preferably introduced or inserted into the pseudodermis (PD) or the pseudodermis preparation in a number or density of 1 to 50/cm² pseudodermis (PD) and more particularly 3 to 7/cm² pseudodermis (PD).

In another embodiment, the introduction or insertion of the pseudopapillae (PP) in step (c) can be carried out by directly injecting or inserting the pseudopapillae (PP), which contain the cultivated dermal papilla cells on a suitable carrier and/or in a matrix, more particularly a gel matrix, into the pseudodermis (PD) or the pseudodermis preparation. In one particular embodiment, the introduction of the pseudopapillae into the pseudodermis preparation takes place before contraction to the pseudodermis is complete. In a particularly preferred embodiment, the introduction or insertion of the pseudopapillae, preferably the injection, can take place as soon as the pseudodermis preparation has formed the matrix (or gel), preferably before or at the beginning of the contraction phase.

Where this procedure is adopted (except where pseudopapillae formed by carriers covered with papilla cells are used), the pseudopapillae (PP) are again preferably introduced or inserted into the pseudodermis (PD) or the pseudodermis preparation in a number or density of 1 to 50/cm² pseudodermis (PD) and, more particularly, 3 to 7/cm² pseudodermis (PD).

In the injection of papilla cells growing on suitable carriers, which represent individual pseudopapillae, preferably 100 to 100,000 pseudopapillae are introduced per cm³ pseudodermis.

Alternatively, the precursors of such pseudopapillae (PP) may also be injected or inserted into the pseudodermis (PD) or the pseudodermis preparation. These precursors consist of a mixture of the cultivated dermal papilla cells and at least one matrix former MPₚₚ, as described above, so that this mixture then forms the matrix and hence the pseudopapillae (PP) in situ in the pseudodermis (PD) or the pseudodermis preparation, more particularly by gelling, i.e. in this embodiment, the pseudopapillae (PP) are formed in situ in the pseudodermis (PD) or the pseudodermis preparation.

In another particularly preferred embodiment, the introduction or embedding of the pseudopapillae (PP) in step (c) may be carried out by directly mixing the pseudopapillae (PP), which contain the cultivated dermal papilla cells on a carrier, with the contractile cells and the matrix-forming, more particularly gel-forming, medium MFMPD, which contains at least one matrix former MFPD, more particularly gel former, and optionally other constituents, during the preparation of the pseudodermis. This early mixing of the pseudopapillae with the pseudodermis preparation leads to a uniform skin/hair equivalent. The pseudopapilla formed by the papilla cells growing on the carriers may be introduced or inserted into the pseudodermis (PD) in a density of 5,000 to 1,000,000 PP/cm³ pseudodermis (PD) and more particularly in a density of 10,000 to 80,000 pseudopapillae (carrier particles)/cm³ pseudodermis (PD). The pseudopapilla formed by the papilla cells growing on the carriers may be introduced or inserted into the pseudodermis preparation in a density of 1,000 to 100,000 PP/cm³ pseudodermis preparation (PD) and more particularly in a density of 1,500 to 60,000 pseudopapillae (carrier particles)/cm³ pseudodermis preparation (PD).

In order realistically to adjust the in vivo system, the introduction or insertion in step (c), more particularly where punches are inserted and the pseudopapillae (PP) or their precursors are injected into the pseudodermis (PD), takes place at an angle of 30° to 90° and more particularly 40° to 60°, based on the plane of the pseudodermis (PD).

The introduction or insertion of the pseudopapillae (PP) into the pseudodermis (PD) may be carried out at regular intervals.

Before or after the introduction or insertion of the pseudopapillae (PP) or their precursors in step (c), a reconstructed epidermis (pseudoepidermis; PE) or a reconstructed periderm (pseudoperiderm; PI) may optionally be applied to the pseudodermis (PD) in step (d). The pseudoepidermis is preferably applied after the introduction or insertion of the pseudopapillae (PP) or their precursors in step (c). The pseudoepidermis (PE) or the pseudoperiderm (PI) may consist of cultivated keratinocytes, hair follicle keratinocytes (ORS and/or matrix keratinocytes), more particularly outer root sheath keratinocytes (ORS keratinocytes) and/or epidermal keratinocytes and optionally of melanocytes, more particularly outer root sheath melanocytes (ORS melanocytes) and/or epidermal melanocytes, and optionally other constituents. The keratinocytes and the melanocytes present, if any, may be applied to the pseudodermis (PD) individually in separate monolayers or multilayers or together in admixture as a monolayer or multilayer. Depending on the medium used, the outer root sheath keratinocytes (ORS keratinocytes) in particular can form a pseudoperiderm (PI). The pseudoperiderm with its periderm-like structure corresponds to the conditions in the embryonic follicle morphogenesis. This has the advantage that the natural conditions, more particularly the direct and indirect cell/cell and/or cell/matrix interactions observed in a three-dimensional geometry, can thus be investigated or influenced.

One embodiment of the process according to the invention may be carried out as follows. To produce the model, a pseudodermis (PD) is first prepared from epidermal fibroblasts in a collagen matrix. “Holes” can then punched into the pseudodermis (PD) at a certain angle (for example 30-90°) and may then optionally be lined with basal membrane proteins (for example laminin, collagen IV, etc.) or, for example, with Matrigel®, a mixture of basal membrane proteins, or mixtures of Matrigel with collagen (more particularly with type I collagen). Reconstructed papillae (pseudopapillae; PP) are then inserted into the “holes”. These reconstructed papillae may be obtained by mixing dermal papilla cells cultivated from hair follicle papillae with a gel, for example Matrigel® or collagen (particularly with collagen type I) or a mixture thereof, and gelling the mix in a small number of passages. After gelation, the pseudopapillae (PP) can be punched from the gel and inserted into the pseudodermis (PD). However, gelation may also take place in situ after introduction into the pseudodermis. Alternatively, however, the dermal papilla cells embedded in Matrigel® may also be directly injected into the pseudodermis (PD) without “holes” having been punched beforehand or the dermal papilla cells embedded in Matrigel® are introduced into the pseudodermis (PD) via a pricking channel.

Finally, a cell layer of hair follicle melanocytes and/or hair follicle keratinocytes (ORS and/or matrix keratinocytes) can be applied to the pseudodermis (PD).

In the process according to the invention, therefore, the papilla is reconstructed from cultivated dermal papilla cells on a suitable carrier and/or a suitable gel former (for example Matrigel®) by introduction or insertion (for example injection, grafting, etc.) into the pseudodermis preparation or the pseudodermis (PD). Optionally, this can also be done by some form of demarcation from the surrounding environment. The reconstructed papillae (pseudopapillae; PP) comprise a combination containing a suitable gel former (for example Matrigel®) and cultivated dermal papilla cells. According to the invention, the papillae are reconstructed, for example, by insertion of so-called pseudopapilla plugs which share the three-dimensional structure of the hair follicle into the pseudodermis (PD), for example by directly injecting dermal papilla cells embedded, for example, in Matrigel® into the reconstructed dermal compartment.

In a preferred embodiment, the pseudopapilla can also be reconstructed by growing papilla cells on a suitable carrier. The papilla cells may then either be inserted into preformed “holes” in the pseudodermis or may simply be integrated therein by pressure or directly mixed with the contractile cells and the gel former (or the pseudodermis preparation) during the production of the pseudodermis.

The pseudopapilla (PP) influences the structure of the optionally overlying pseudoepidermis (PE) or the pseudoperiderm (PI) of keratinocytes and, optionally, melanocytes. A hair-follicle-like structure is formed under these conditions. The model produced in this way may be used, for example, to study substances (pharmacological, cosmetic, etc.) for hair growth and hair structure and to study the effect of substances on hair pigmentation.

The present invention also relates to the hair/skin equivalent obtainable by the process according to the invention.

The skin/hair equivalent according to the invention is in particular a skin/hair model with, in particular, three-dimensionally formed, optionally spatially demarcated, reconstructed papillae (pseudopapillae; PP) with follicle-like or follicular structures, including those reminiscent of the earliest stages of hair morphogenesis, (i.e. morphogenesis stages I to III), the skin/hair equivalent comprising a reconstructed dermis (pseudodermis; PD) into which the pseudopapillae (PP) are introduced or inserted, the pseudopapillae (PP) comprising cultivated papilla cells, more particularly dermal papilla cells (hair papilla cells), on a suitable carrier and/or in a suitable matrix, more particularly gel matrix, and a reconstructed epidermis (pseudoepidermis; PE) or a pseudoperiderm (PI) optionally being applied to the pseudodermis (PD).

Accordingly, the skin/hair equivalent, more particularly the hair follicle model, according to the invention generally comprises a pseudodermis (PD) with dermal papillae reconstructed therein (pseudopapillae; PP). One or more layers of keratinocytes, which form or have formed themselves into a pseudoepidermis (PE) or a pseudoperiderm (PI), and optionally one or more layers of melanocytes can be applied over the pseudodermis (PD). This in vitro model is suitable, for example, for effectiveness and compatibility tests in the pharmaceutical, medical and cosmetics fields. Follicle-like or follicular structures, including those reminiscent of the earliest stages of hair morphogenesis (morphogenesis stages I to III), are formed in the three-dimensional hair/skin model according to the invention.

The present invention also relates to the use of the skin/hair equivalent according to the invention as described in claims 39 to 42. In addition, for further details, reference may be made to the foregoing observations on the process according to the invention and the skin/hair equivalent according to the invention which also apply accordingly to the use according to the invention.

The present invention also relates to a system, more particularly a test system (for example a screening system), which comprises the skin/hair equivalent according to the invention. In addition, for further details, reference may be made to the foregoing observations on the process according to the invention, the skin/hair equivalent according to the invention and the use according to the invention which also apply accordingly to the system according to the invention.

The present invention affords a number of advantages. The skin/hair equivalent according to the invention is a reconstructed model which is more standardizable than the isolated hair follicle. It reduces the demand for hair follicles and is closer to the in vivo situation than monolayer systems. In addition, it is an alternative to animal tests.

The reconstructed model according to the invention is a complex three-dimensional model which simulates the hair follicle in vivo in its structure and its histological composition, resulting in a high level of relevance of the information provided on the effectiveness and compatibility of active substances (cosmetics, Pharmaceuticals, etc.).

The following end points inter alia can be evaluated or measured to obtain information on the effectiveness of substances in regard to an improvement in hair structure and the influencing of hair growth: proliferation/apoptosis of the keratinocytes via the pseudopapilla; structure and arrangement of the keratinocytes via the pseudopapilla; structure of the epidermis; structure of the stratum corneum; volume and structure of the dermal papilla; analysis of certain hair-specific proteins (more particularly hair-specific keratins); analysis of cytokines, chemokines and all kinds of messenger substances formed inter alia by the dermal papilla; hair array analysis, proteom or expression analyses, etc.

The reconstructed hair follicle model according to the invention is the only reconstructed hair follicle model with which influences on hair pigmentation can be measured (for example pigmentation of the amelanocytic ORS melanocytes; melanin synthesis; melanin granula; arrangement of the melanocytes; migration of the melanocytes; modification of melanocyte markers, such as TRP-1, TRP-2, NKI/beteb, etc.; release of melanin to keratinocytes). It may also be used, for example, for hair array analysis.

The hair/skin model according to the invention is suitable for various applications in the medical, pharmaceutical and cosmetics fields (for example for the discovery of active substances with a biological effect on the hair follicle by influencing hair pigmentation, hair growth and hair structure, in in vitro test systems, in screening processes, for the development of cosmetic products, etc.). The model or equivalent according to the invention provides information on the effect of substances on hair follicle cells with in vivo relevance. The model or equivalent according to the invention provides hair follicles or parts of hair follicles in a three-dimensional model. In contrast to isolated hair follicles, the reconstructed papillae (pseudopapillae; PP) are available at any time and standardizable. Dermal papillae which share the three-dimensional structure of the hair follicle are reconstructed. It is possible in this way to evaluate how applied active substances act on the structure of the reconstructed three-dimensional model and what can be read into this with regard to the effect of these substances on the hair follicle (for example hair growth, hair structure, hair pigmentation, etc.).

Other embodiments, modifications and variations of the present invention will be readily apparent to the expert on reading the present specification and can be put into practice without departing from the scope of the invention.

The following Examples are intended to illustrate the invention without limiting it in any way.

### EXAMPLES

The production of a skin/hair model with reconstructed dermal papillae (pseudopapillae; PP) embedded in or inserted into a pseudodermis (PD) is described in the following. To this end, dermal papilla cells are either injected into the pseudodermis (PD), or placed by means of punches in the pseudodermis (PD). Alternatively, dermal papilla cells grown on suitable carriers can be embedded by mixing with the pseudodermis preparation. The pseudodermis (PD) may subsequently be covered with a layer of epidermal or hair follicle keratinocytes, which represent a pseudoepidermis (PE) or a pseudoperiderm (PI), and optionally with melanocytes.

ABBREVIATIONS

| | |
|---|---|
| DMEM | Dulbecco's Modified Eagle Medium |
| DP | dermal papilla |
| DPC | dermal papilla cells |
| ECM | extracellular matrix |
| EGF | epidermal growth factor |
| PCS | fetal calf serum |
| FGF | fibroblast growth factor |
| HBSS | Hank's buffered salt solution |
| NCAM | neural cell adhesion molecule |
| NHEK | normal human epidermal keratinocytes |
| ORS | outer root sheath keratinocytes |
| RPMI | RPMI Medium was developed by Moore et Roswell |
| | Park Memorial Institute, hence the RPMI |
| TGF | transforming growth factor |

Procedure and Methods: Preparation of the Single Cell Cultures
1. Dermal Fibroblasts

Dermal fibroblasts are isolated from human foreskin. The epidermis and dermis of the foreskin are enzymatically separated from one another with thermolysin (0.5 mg/nl HEPES buffer). To extract the fibroblasts, the dermis is digested (3-4 h at 37° C.) with collagenase H (0.2 U/ml, Boehringer Mannheim, Mannheim, Germany). After the incubation phase, the solution is carefully mixed to thin out the cells, filtered through a cell sieve and the cells are centrifuged off. Cultivation is carried out in Dulbecco's Modified Eagle Medium (DMEM) with Glutamax I (L-alanyl-L-glutamine) and sodium pyruvate, 4,500 mg L⁻¹ glucose and pyridoxine (Gibco BRL, Karlsruhe, Germany) enriched with 10% fetal calf serum (FCS) (Gibco BRL, Karlsruhe, Germany), 25 ug ml⁻¹ gentamicin (Sigma, Taufkirchen, Germany) and 100 Ul mL⁻¹ penicillin G (Sigma), as described in K. Schlotmann et al., Cosmetic Efficacy claims in vitro Using a 3d Human Skin Model, Intl. J. Cosmet. Sci., 23:310-319 (2001).

2. Epidermal Keratinocytes

Dermal fibroblasts are isolated from human foreskin. The epidermis and dermis of the foreskin are enzymatically separated from one another with thermolysin (0.5 mg/nl HEPES buffer). To extract the keratinocytes, the epidermis is digested (20 mins. at 37° C.) with trypsin (Gibco BRL, Karlsruhe, Germany). After the incubation phase, the solution is carefully mixed to thin out the cells, filtered through a cell sieve and the cells are centrifuged off. Cultivation is carried out in a mixture of DMEM Glutamax I and Ham's F 12 (Sigma) (3:1) enriched with newborn calf serum (NCF, fetal clone II, Hyclone), epidermal growth factor (EGF) (Sigma), insulin (Sigma), hydrocortisone (Sigma), triiodo-L-thyronine (Sigma), adenine (Sigma), cholera toxin (Sigma) and antibiotics on feeder layers (dermal fibroblasts irradiated with 60 gray to inhibit proliferation), as described in K. Schlotmann et al., Cosmetic Efficacy claims in vitro Using a 3d Human Skin Model, Intl. J. Cosmet. Sci., 23:310-319 (2001).

3. Outer Root Sheath Keratinocytes (ORS Keratinocytes)

ORS keratinocytes are isolated from human hairs plucked from the back of the head. To extract the keratinocytes, the remains of the hair bulb are first removed with a scalpel and the follicles digested (40 mins. at 37° C.) with trypsin (protease from Gibco BRL, Karlsruhe, Germany). After the incubation phase, the solution is carefully mixed to thin out the cells, filtered through a cell sieve and the cells are centrifuged off. Cultivation is carried out in a mixture of DMEM Glutamax I and Ham's F 12 (Sigma) (3:1) enriched with newborn calf serum (NCF, fetal clone II, Hyclone), epidermal growth factor (EGF) (Sigma), insulin (Sigma), hydrocortisone (Sigma), triiodo-L-thyronine (Sigma), adenine (Sigma), cholera toxin (Sigma) and antibiotics on feeder layers (dermal fibroblasts irradiated with 60 gray to inhibit proliferation), as described in K. Schlotmann et al., Cosmetic Efficacy claims in vitro Using a 3d Human Skin Model, Intl. J. Cosmet. Sci., 23:310-319 (2001).

4. ORS Melanocytes are Isolated by Tobin et al.'s Method

See D. J. Tobin et al., Isolation and Long-Term Culture of Human Hair-Follicle Melanocytes, J. Invet. Dermatol., 104:86-89 (1995).

5. Dermal Papilla Cells

Dermal papilla cells are isolated from scalp (temporal or occipital region) with intact hair follicles. To this end, the upper dermis is removed and the follicles together with the dermal papilla are plucked from the dermis using watchmaker's tweezers. The further isolation of the dermal papilla is carried out under a stereo magnifying glass. The dermal papilla is then transferred to a culture bottle coated with collagen I with the aid of a microcapillary. Cultivation is carried out either in RPMI 1640 Medium containing glutamine (Sigma) enriched with 20% fetal calf serum (FCS) (Gibco BRL, Karlsruhe, Germany) and antibiotics or in Chang's Medium containing 10% fetal calf serum, as described in R. Warren et al., Improved Method for the Isolation and Cultivation of Human Scalp Dermal Papilla Cells, J. Invest. Dermatol., 98:693-699 (1992).

6. Production of the Pseudodermis (PD)

To establish the hair model, the pseudodermis (PD) is first developed. To this end, dermal fibroblasts of the fourth passage are mixed with collagen I from rat tail tendon and sown in multiwell plates. The object of this is to optimize the number of cells, the cultivation time, the layer thickness and the size of the dermis equivalent (pseudodemmis; PD). Production is carried out to the following protocol: 1 part of HBSS buffer (Gibco BRL) is mixed with 8 parts of collagen solution (Becton Dickinson) and neutralized with 1 M sodium hydroxide. The required quantity of cells is added in 1 part of fetal calf serum (FCS, Gibco BRL). The mixture obtained (pseudodermis preparation) is poured into cell culture dishes and incubated for 1 h at 37° C. in an incubation cabinet. After polymerization of the collagen, the models are covered with DMEM supplemented with 10% FCS and penicillin/streptomycin. The medium is changed three times per week over a period of seven days.

The following preparations were tested:

| Parameter | Preparation |
|---|---|
| Cell density | 1 × 1 0^{&} cells/ml gel preparation/2.5 × 10⁵ cells/ml |
| | gel preparation |
| Cultivation time | 7 days/14 days |
| Layer thickness | 4 mm; 5.2 mm; 6 mm; 8 mm; 10 mm |
| of model | |
| Size | 24 wells (0 1.6 cm), 12 wells (0 2.2 cm) |

7. Optimizing the Monolayer Culture of Dermal Papilla Cells (DPC)

To optimize the monolayer culture of dermal papilla cells, two different media are used to prepare the primary cultures.

1. RPMI 1640 (Sigma) containing 20% PCS with penicillin/streptomycin

2. Chang Medium (Irvine Scientific) containing 10% PCS

On the one hand, the primary cultures (PO) are prepared in the media shown, on the other hand a test is conducted to determine whether the medium used to continue the cultures has an effect on the proliferation and the morphology and arrangement of the cells.

8. Influence of Matrigel® on the Proliferation of the Dermal Papilla Cells

Matrigel® (Becton Dickinson) may be used as the surrounding medium for the dermal papilla cells placed in the pseudodermis (PD)[4,5]. Matrigel® is obtained from the Engelbreth-Holm-Swarrn Tumor of mice and mainly contains laminin, collagen IV, heparan sulfate proteoglycans, Tissue Plasmimogen Activator, nidogen (more particularly entactin) and also TGF-β, FGF and other growth factors of the EHS Tumor. In order to study the influence of Matrigel® on the proliferation of the DPC, primary cultures of DP are carried out in cell culture bottles coated with Matrigel® and growth is observed. For comparison, DP are simultaneously grown in cell culture bottles coated with collagen I.

9. Preparation of the Injection Channels for Insertion of the DPC into the Pseudodermis (PD)

To prepare the injection channels, a button cannula, a conventional cannula and a 2 mm punch are used. To show up the channels, a solution of 10% Berlin Blue in 1% agarose solution is prepared and injected into the dermis equivalent using the button cannula and the conventional cannula. Where the punches are used, the channels prepared with the biopsy punches are filled either immediately or after 24 h using the button cannula. The channels are prepared with the aid of a stereo magnifying glass. After 24 h, the models are deep-frozen to prepare cryosections and for histological examination.

10. Production of the Reconstructed Papilla by Insertion of Punches into the Pseudodermis (PD)

First, dermal papilla cells of the second passage were mixed in a concentration of 250,000 cells/ml with collagen I from rat tail tendon and sown in multiwell plates. The number of cells was selected in line with the production of the pseudodermis (PD).

Production was carried out to the following protocol: 1 part of HBSS buffer, (Gibco BRL) was mixed with 8 parts of collagen solution (Becton Dickinson) and neutralized with 1 M sodium hydroxide. The required quantity of cells was added in 1 part of fetal calf serum (FCS, Gibco BRL). The mixture obtained was poured into cell culture dishes and incubated for 1 h at 37° C. in an incubating cabinet. After polymerization of the collagen, the models were covered with Chang Medium supplemented with 10% FCS. The medium was changed three times per week over a period of eight days.

After this cultivation period, holes were made in a 7-day-old pseudodermis with the aid of a 2 mm punch. Quantities of 5 ul of Matrigel were injected and 3 mm biopsies from the polymerized collagen/DPC mixture inserted into the holes thus formed. The models were covered with Chang medium supplemented with 10% FCS and the medium was changed three times per week over a period of six days. The models were then subjected to histological and immunohistochemical evaluation.

Since collagen I in the dermal papilla is not expressed in the hair follicle and since pure Matrigel® cannot be punched, an attempt was made in another experiment to create a more physiological environment for dermal papilla cells. To this end, dermal papilla cells of the second passage were mixed in a concentration of 250,000 cells/mL with a combination of collagen I from rat tail tendon and Matrigel® and sown in multiwell plates. In the production process, the collagen normally used in the preparation of the gels was replaced by collagen/Matrigel® mixed in various ratios.

In further experiments, other punch models were prepared with an increased cell concentration (between 500,000 cells/ml and 2×106 cells/ml) and the combination found to be optimal of 1 part Matrigel® and 2 parts collagen I. After completion, the models were subjected to histological and immunohistochemical evaluation.

11. Production of the Reconstructed Papilla by Injection of Dermal Papilla Cells into the Pseudodermis (PD) or the Pseudodermis Preparation

To establish very high cell densities, the cells were first mixed with Matrigel® in the required cell concentration and then centrifuged off in a refrigerated centrifuge (5 mins, 1,000 r.p.m., T=1° C.). The excess Matrigel® was then removed, the cell pellet left behind was taken up with a pipette and directly injected into the pseudodermis (PD) or the pseudodermis preparation. The models thus produced were subjected to histological and immunohistochemical evaluation.

12. Production of the Reconstructed Papilla by Embedding of Microcarriers Overgrown with Dermal Papilla Cells in the Pseudodermis (PD) or the Pseudodermis Preparation

The use of microcarriers enables dermal papilla cells to be cultivated in a predetermined, reproducible three-dimensional structure which effectively simulates the physiological constellation in the hair follicle. In principle, suitable microcarriers are any type of three-dimensional carriers on which the dermal papilla cells can be cultivated. The following carrier materials were tested:

| Microcarrier* | Specification | Manufacturer |
|---|---|---|
| Siran | Porous glass carrier, 0.4-0.7 mm | Schott |
| Ashby | Porous silicone carrier with | Ashby Scientific |
| | steel particles 0.8 × 0.25 mm | |
| Cultisphere S and G | Gelatine matrix, 130-380 um | Percell |
| Cytodex 1 and 3 | Dextran matrix, | Amersham |
| | 140-250 um | Bioscience |
| Biosilon | Nonporous polystyrene matrix, | Nunc |
| | 0 160-300 urn | |
| 2D MicroHex | Nonporous polystyrene matrix, | Nunc |
| | 125 × 25 urn | |
| Ca alginate | Nonporous material, | Own make |
| | 0 ca. 1.5 mm | |

Before the microcarriers are inserted into the pseudodermis or the pseudodermis preparation, they are preincubated with dermal papilla cells so that they grow in sufficient density. Depending on the carrier selected, cultivation may be carried out in a shaken or stirred culture in a stirred or fluidized bed reactor (Eddy pro 10, Papaspyrou Biotechnologie) or in spinner bottles. The dermal papilla cells were always cultivated for several days in Chang Medium. In that time, cell growth was monitored by a neutral red coloration, MTT test, determination of the cell count and by immunohistochemical detection (for example expression of Versican) and the suitability of the various carriers was thus established.

It is also possible to produce nonporous calcium alginate beads containing dermal papilla cells. To this end, a liquid sodium alginate solution is mixed with dermal papilla cells and the resulting mixture is introduced dropwise through a cannula into a calcium chloride solution. The sodium ions are replaced by the calcium ions, resulting in a polymerization of the alginate in which the papilla cells are incorporated in the alginate.

Before insertion into the injection channels of the pseudodermis (PD) or the pseudodermis preparation (more particularly with the already formed matrix), the carriers were partly coated with Matrigel®. Instead or in addition, the channels in which the microcarriers were inserted can also be lined with Matrigel®.

Besides the injection of the microcarriers into the pseudodermis or the pseudodermis preparation with the matrix formed, the embedding of the carriers overgrown with dermal papilla cells in the pseudodermis during its production (i.e. the pseudodermis preparation) was also investigated. To this end, various volumes of microcarriers overgrown with dermal papilla cells were mixed with the dermal fibroblasts and the collagen I and the models were cultivated for 7 days.

13. Production of a Skin Model of a Pseudodermis and an Epidermis of Epidermal Keratinocytes (NHEK) or a Pseudoperiderm of Hair Keratinocytes (ORS keratinocytes) (with and without Pseudopapilla)

In order further to develop the reconstructed hair follicle model, the pseudodermis was transferred to Snapwell Inserts (Corning Costar) after five days' culture and was first covered with epidermal keratinocytes (500,000 cells/model). After one week's submerse cultivation in keratinocyte medium (DMEM Glutamax I and Hams'F12 (Sigma) (3:1) enriched with newborn calf serum (NCF, fetal clone II, Hyclone), epidermal growth factor (EGF) (Sigma), insulin (Sigma), hydrocortisone (Sigma), triiodo-L-thyronine (Sigma), adenine (Sigma), cholera toxin (Sigma), ascorbyl-2-phosphate (Sigma) and antibiotics, the models were transferred to the air/liquid interface and cultivated for another two weeks in DMEM Glutamax I and Hams'F12 (Sigma) (3:1) enriched with insulin (Sigma), hydrocortisone (Sigma), ascorbyl-2-phosphate (Sigma), bovine serum albumin (BSA) (Sigma) and antibiotics.

In another experiment, the epidermal keratinocytes were replaced by ORS keratinocytes from the hair follicle which were sown with 800,000 cells/model. Cultivation (submerse) was carried out with keratinocyte medium for 7 days.

Results

The following parameters were found to represent optimal conditions for the production of the pseudodermis (PD): 2.5×10⁵ cells/ml gel preparation (pseudodermis preparation) are cultivated for 7 days in a 12-well plate. The layer thickness at the time of sowing is 10 mm. After 7 days, the model has completely contracted, the remaining layer thickness being 3.3 mm which corresponds to a contraction of ca. 67%.

In the preparation of the DPC primary cultures, the proliferation of the cells was found to be better in Chang Medium than in RPMI Medium. When the cultures were continued, there were no differences in cell growth between the two media. Where both media are used, the cells are typically arranged in clusters both in the primary culture and in the first passage.

Where culture bottles coated with Matrigel® were used, cell growth was slower by comparison with growth surfaces coated with collagen I. This is presumably attributable to the high content of ECM molecules in Matrigel® which promotes cell differentiation in the event of slow cell growth.

A button cannula may be used to prepare the injection channels because a sufficiently large channel can be prepared with such a cannula without piercing the pseudodermis (PD). It is also possible to use punches, in which case the channels can be filled with the cell/Matrigel® mixture or punches of Matrigel® with DPC can be placed in the existing channels.

The production of the pseudopapilla by the embedding of microcarriers overgrown with dermal papilla cells by injection or by mixing with the gel preparation (or the pseudodermis preparation) is particularly suitable. Cultivation on Siran carriers in a fluidized bed reactor and, more particularly, shaking cultivation on Percell Cultispheres® can be carried out with particular advantage. Dense cell growth on the surfaces was obtained in a few days.

Coating of the pseudodermis with ORS keratinocytes in keratinocyte medium (cf. appendix) leads to the formation of a pseudoperiderm whereas coating with epidermal keratinocytes (NHEK) and keratinocytes in air/liquid interface medium leads to the formation of an epidermis.

Fibroblast Medium

| | |
|---|---|
| DMEM | |
| FCS | 10% |
| Ascorbyl-2-phosphate | 1 mM |
| Penicillin G | 100 Ul/ml |
| Gentamicin | 25 ug/ml |

Keratinocyte Medium

| | |
|---|---|
| DMEM/Nutrient Mixture Ham's F12 | 3:1 |
| Fetal Clone II | 10% |
| EGF | 10 ng/ml |
| Hydrocortisone | 0.4 ug/ml |
| Insulin | 0.12 Ul/ml |
| Cholera toxin | 10⁻¹⁰ M |
| Triiodothyronine | 2 * 10⁻⁹ M |
| Adenine | 24.3 ug/ml |
| Penicillin G | 100 Ul/ml |
| Gentamicin | 25 ug/ml |
| Ascorbyl-2-phosphate | 1 mM |

RPMI-1640-Medium (Gibco, BRL, Karlsruhe)

| | |
|---|---|
| RPMI 1640 with L-glutamine | |
| FCS | >20% |
| Penicillin | 100 Ul/ml |
| Streptomycin | 100 ug/ml |

Chang Medium (Irvine Scientific, Santa Ana, USA)

| Chang Medium D | |
|---|---|
| FCS | 10% |

Composition Of Chang Medium D

| | |
|---|---|
| Salts | 8515 mg/ml |
| Dextrose | 880 mg/ml |
| Amino acids | 802 mg/mi |
| L-glutamine | 259 mg/ml |
| Polypeptides | 189 mg/ml |
| Vitamins | 61 mg/ml |
| Deoxyribonucleosides | 35 mg/ml |
| Ribonucleosides | 35 mg/ml |
| Sodium pyruvate | 97 mg/ml |
| Steroidal hormones | 0.0009 mg/ml |
| BSA Bovine serum proteins | 12% |
| Other components | 9 mg/ml |

Medium for the Air/Liquid Interface

| | |
|---|---|
| DMEM/nutrient mixture Ham's F12 | 3:1 |
| Hydrocortisone | 0.4 ug/ml |
| Insulin | 0.12 Ul/ml |
| Penicillin G | 100 UI/ml |
| Gentamicin | 25 ug/ml |
| BSA (bovine serum proteins) | 1.6 mg/ml |
| Ascorbyl-2-phosphate | 1 mM |

### APPENDIX II: LITERATURE REFERENCES CITED IN THE EXAMPLE

K. Schlotmann et al., Cosmetic Efficacy claims In vitro Using a 3D Human Skin Model, Int. J. Cosmet. Sci. 23, 310-319 (2001).
R. Warren et al., Improved Method for the Isolation and Cultivation of Human Scalp Dermal Papilla Cells, J. Invest. Dermatol. 98, 693-699 (1992).
D J. Tobin et al., Isolation and Long-Term Culture of Human Hair-Follicle Melanocytes, J. Invest. Dermatol. 104, 86-89 (1995).
A. Limat et al., Outer root sheath cells organize into epidermoid cyst-like spheroids when cultured in Matrigel®. Cell Tissue Res. 275, 169-176 (1994).

1. An artificial skin and hair substitute, comprising a pseudodermis comprising cultivated contractile cells in a first matrix and, in contact with the pseudodermis, pseudopapillae comprising cultivated papilla cells in a second matrix.

2. The artificial skin and hair substitute of claim 1 further comprising a pseudoepidermis surmounting the pseudodermis.

3. The artificial skin and hair substitute of claim 1 further comprising a pseudoperioderm surmounting the pseudodermis.

4. The artificial skin and hair substitute of claim 1 wherein said contractile cells are fibroblasts.

5. The artificial skin and hair substitute of claim 4 wherein said fibroblasts are dermal fibroblasts.

6. The artificial skin and hair substitute of claim 1 wherein said second matrix is collagen.

7. The artificial skin and hair substitute of claim 1 wherein said second matrix is type I collagen, type III collagen, or mixtures thereof.

8. The artificial skin and hair substitute of claim 1 wherein said cultivated papilla cells are dermal papilla cells

9. The artificial skin and hair substitute of claim 1 wherein said pseudopapillae further comprise a carrier comprising polysaccharide- or polypeptide-based material.

10. The artificial skin and hair substitute of claim 9 wherein said carrier is dextran or gelatin.

11. The artificial skin and hair substitute of claim 9 wherein said carrier is spherical.

12. The artificial skin and hair substitute of claim 9 wherein the surface of said carrier is modified.

13. The artificial skin and hair substitute of claim 12 wherein said surface of said carrier is coated with matrix forming proteins, matrix, scleroproteins, tissue plasminogen activator, Matrigel®, or mixtures thereof.

14. The artificial skin and hair substitute of claim 12 wherein said surface of said carrier is coated with collagen.

15. The artificial skin and hair substitute of claim 12 wherein said surface of said carrier is coated with type I collagen, type II collagen, or type IV collagen.

16. The artificial skin and hair substitute of claim 12 wherein said surface of said carrier is coated with laminin, gelatin, chitosan, glucosamine, glycosaminoglycans, heparan sulfate proteoglycan, sulfated glycoprotein, or mixtures thereof.

17. The artificial skin and hair substitute of claim 9 wherein said carrier has a size in the range of from about 50 μm to about 2000 μm.

18. The artificial skin and hair substitute of claim 9 wherein said carrier has a size in the range of from about 100 μm to about 500 μm.

19. The artificial skin and hair substitute of claim 1 wherein pseudopapillae are formed in situ in said pseudodermis.

20. The artificial skin and hair substitute of claim 1 wherein said pseudopapillae are present at a density of about 3/cm² to about 50/cm².

21. The artificial skin and hair substitute of claim 1 wherein said pseudopapillae are present at a density of about 3/cm² to about 7/cm².

22. The artificial skin and hair substitute of claim 2 herein said pseudoepidermis comprises cultivated keratinocytes, hair follicle keratinocytes, epidermal keratinocytes, melanocytes, or combinations thereof.

23. The artificial skin and hair substitute of claim 3 herein said pseudoperiderm comprises cultivated keratinocytes, hair follicle keratinocytes, epidermal keratinocytes, melanocytes, or combinations thereof.

24. The artificial skin and hair substitute of claim 22 wherein said hair follicle keratinocytes are outer root sheath keratinocytes, matrix keratinocytes or combinations thereof.

25. The artificial skin and hair substitute of claim 22 wherein said melanocytes are outer root melanocytes, epidermal melanocytes, or combinations thereof.

26. The artificial skin and hair substitute of claim 23 wherein said hair follicle keratinocytes are outer root sheath keratinocytes, matrix keratinocytes or combinations thereof.

27. The artificial skin and hair substitute of claim 23 wherein said melanocytes are outer root melanocytes, epidermal melanocytes, or combinations thereof.

28. A method of producing an artificial skin and hair substitute comprising:
contacting a pseudodermis comprising cultivated contractile cells in a first matrix with a pseudopapillae comprising cultivated papilla cells in a second matrix.

29. The method of claim 28 wherein said contractile cells are mixed with a matrix-forming medium containing at least one matrix former.

30. The method of 29 wherein said matrix-forming medium is type I collagen, type III collagen, or combinations thereof.

31. The method of claim 28 wherein a nutrient medium is applied to said pseudodermis.

32. The method of claim 28 further comprising forming cavities in said pseudodermis and placing said pseudopapillae therein.

33. The method of claim 32 further comprising lining said cavities with at least one of collagen or matrix protein.

34. The method of claim 32 wherein said cavities have a diameter in the range of about 0.5 mm to 4 mm.

35. The method of claim 32 wherein said cavities have a diameter of about 2 mm.

36. The method of claim 33 wherein said collagen is type IV collagen or said matrix protein is laminin.

37. The method of claim 28 wherein said pseudopapillae are placed into said pseudodermis at an angle of about 30° to about 90°.

38. The method of claim 28 wherein said pseudopapillae are placed into said pseudodermis at an angle of about 40° to about 60°.

39. A method for testing the compatibility of a product, comprising:
providing an artificial skin and hair substitute of claim 1;
contacting said artificial skin and hair substitute with the product; and
determining the compatibility of the product with the artificial skin and hair substitute.

40. The method of claim 39 wherein determining the compatibility of the product with the artificial skin and hair substitute further comprises determining the compatibility of a hair follicle, hair pigmentation, hair growth, hair color, or combinations thereof.

41. A method for testing the effectiveness of a product, comprising:
providing an artificial skin and hair substitute of claim 1;
contacting said artificial skin and hair substitute with the product; and
determining the effect of the product on the artificial skin and hair substitute.

42. The method of claim 41 wherein the effect of the product on the artificial skin and hair substitute further comprises determining the effect on a hair follicle, hair pigmentation, hair growth, hair color, or combinations thereof.

43. A method for producing a skin and hair equivalent, comprising:
(a) providing a pseudodermis or a pseudodermis preparation;
(b) providing (i) pseudopapillae comprising cultivated dermal papilla cells on a suitable carrier or in a suitable matrix, or (ii) pseudopapillae precursors comprising cultivated dermal papilla cells, in a suitable matrix-forming, medium which is capable of forming a matrix in situ;
(c) introducing the pseudopapillae or pseudopapillae precursors into the pseudodermis or the pseudodermis preparation;
(d) optionally applying a pseudoepidermis or a pseudoperiderm to the pseudodermis.
